(19) United States
(12) Patent Application Publication
(10) Pub. No.: US 2016/0029880 A1
(43) Pub. Date: Feb. 4, 2016

(54) CYSTOSCOPIC DEVICE AND METHODS FOR OPERATING SAME

(71) Applicant: Anil Kumar, Rochester Hills, MI (US)

(72) Inventor: Anil Kumar, Rochester Hills, MI (US)

(21) Appl. No.: 14/813,774

(22) Filed: Jul. 30, 2015

Related U.S. Application Data
(60) Provisional application No. 62/030,934, filed on Jul. 30, 2014.

(51) Int. Cl. A61B 1/12; A61B 1/07; A61B 1/00; A61B 1/307

(52) U.S. Cl. CPC A61B 1/12; A61B 1/307; A61B 1/07; A61B 1/00195

(57) ABSTRACT

A cystoscope device and method for operating and using same are disclosed. The cystoscope comprises a sheath and a bridge removably connected thereto. The bridge includes an offset lens assembly including an upper eyepiece; a light source, and a fiber optic connected to a light source. The lens or eyepiece enables observation within a urological organ while contemporaneously conducting a urological procedure.

### CROSS-REFERENCE TO RELATED APPLICATION

This application is a completion application of co-pending U.S. Provisional Patent Application Ser. No. 62/030,934, filed Jul. 30, 2014 for “Cystoscopic Device and Methods for Operating Same”, the entire disclosure of which is hereby incorporated by reference, including the drawing.

### BACKGROUND OF THE INVENTION

1. Field of the Invention

This present invention relates to cystoscopy procedures and, more particularly, to cystoscope devices and methods for operating same.

2. Prior Art

Known cystoscopes are typically used for diagnostic and surgical medical procedures such as a cystoscopy. Cystoscopes are used to examine the urinary bladder and the interior urethra wall. Typically, cystoscopes have a flexible insertion tube that is inserted into a patient during a cystoscopy procedure. During insertion, the position of the distal end of the insertion tube is controlled by control devices on the cystoscope's handle at the proximal end of the insertion tube.

Known cystoscopes have channels that extend along the length of the cystoscope and come into contact with body tissues and fluids during the procedure. These channels allow air insufflation into a body cavity, water flow to wash the cystoscope's tip, suction through the tip, and biopsies to be taken. The channels must be sufficiently flexible to bend with the insertion tube through the short radius turns. The channels also must substantially maintain their cross-sectional shape and resist kinking to assure continuous air flow, water flow, suction, and biopsy capabilities throughout the procedure.

A cystoscopy is an uncomfortable procedure utilized for the unambiguous diagnosis of bladder cancer, cleansing procedures, and surgical operations including abnormality removal. Cystoscopy enables the identification of abnormalities that may be missed by other diagnostic modalities such as ultrasound, computed tomography, or magnetic resonance imaging. In some cystoscopy devices a lighting instrument is integrally connected to view inside the bladder and urethral wall during insertion. Medical professionals may begin the procedure by cleansing the urethra using a flow of fluid or retrograde pyelogram. In performing a retrograde pyelogram through the cystoscope, a small plastic tube is inserted into the ureter, and contrast material is injected into the ureter and kidney. After cleansing, a typical cystoscopy procedure involves the use of various instruments inserted individually through the channel. A typical cleansing maneuver includes: (1) channeling a camera device through the channel; (2) identifying an abnormality; (3) positioning the cystoscope to air removal; (4) removing the camera device; and (5) inserting a surgical device to remove the abnormality such as a suctioning device or scrapper.

In a conventional cystoscope a suctioning instrument or other device (not shown) is removably mounted at the opening portion 32 for traversal or through the hollow instrument channel out the distal opening portion 13 and the lens is removed.

Prior to the present invention, a lens or camera and suction instruments would be separately inserted, requiring an operator to alternate between instruments. For example, a surgeon-operator would view tissue until finding an abnormality. At this point the surgeon-operator would remove the camera or lens device from the sheath, which remains in place within the urethra. The surgeon-operator subsequently inserts the desired surgical tool, e.g., a suction instrument 40, into the sheath for removal or the identified abnormality and repeats by removing the surgical tool and reinserting the camera device. This interruption of the surgical procedure must be performed frequently when known cystoscopes are used. These periodic interruptions produce dangerous difficulties such as temporary loss of visual orientation, prolonged bleeding and extended operative time.

Thus, a conventional cystoscope does not permit a simultaneous procedure and observation.

As the cystoscopy is an uncomfortable and often painful procedure, any temporal efficiency achieved would be advantageous. Hence, there is a need to decrease procedure time using a cystoscope having a dual camera and instrument capability.

### SUMMARY OF THE INVENTION

A cystoscope device and method for operating and using same are disclosed. The cystoscope comprises a sheath and a bridge removably connected thereto. The bridge includes an offset lens assembly including an upper eyepiece; a light source, and a fiber optic connected to the light source. The lens or eyepiece enables observation within a urological organ.

For a more complete understanding of the present invention, reference is made to the following detailed description and accompanying drawing. In the drawing like reference characters refer to like parts throughout the several views, in which:

### BRIEF DESCRIPTION OF THE DRAWINGS

FIG. 1 is a perspective view of a prior art, conventional cystoscope; and

FIG. 2 illustrates a side view of a cystoscope, partly in cross-section, in accordance with the present invention.

### DESCRIPTION OF THE INVENTION

As is known to the skilled artisan, endoscopic instruments are widely used in urology and are introduced through the urethra to perform various medical procedures at the operative field such as the bladder, prostate or urethra. Typical instruments being the resectoscope, urethro-cystoscope, lithotriptor, and biopsy endoscopic forceps. For ease of description and illustration, the specification herein will disclose and describe the invention in specific connection with a cystoscope and, therefore, should not be limited thereby.

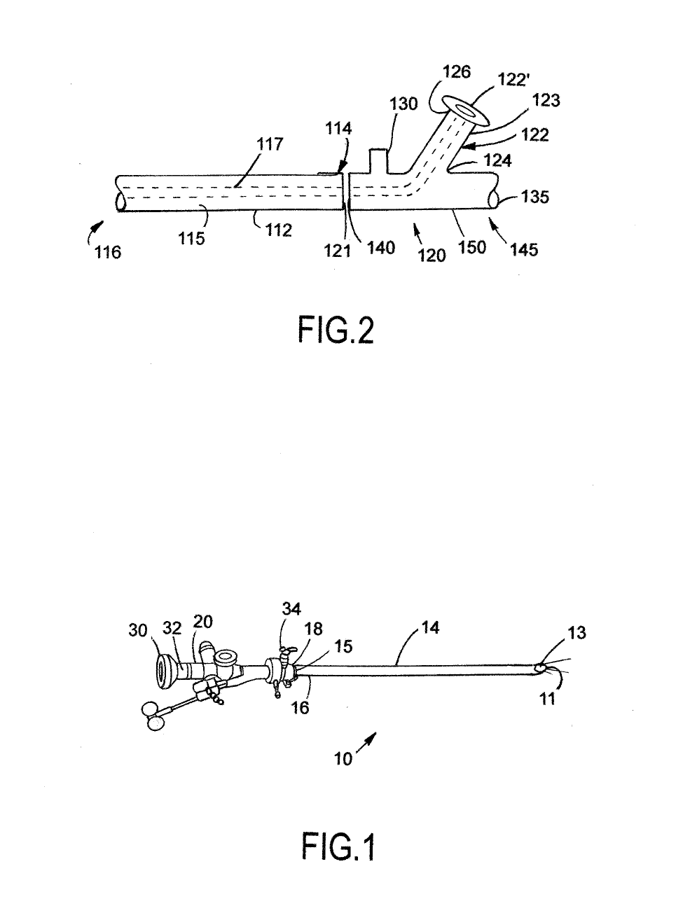

Referring now to the drawing and, in particular, FIG. 1 there is depicted a conventional cystoscope 10. The cystoscope 10 includes a tubular sheath 14 which provides a passageway through the human urethra to the area of visual and operative interest. The sheath 14 includes a hollow inserting portion 12 at its distal end 13 and a proximal main body 16. The distal end 13 of the inserting portion 12 has a distal opening 11. At its proximal end 15 the sheath has a socket base 18 to which are secured a bridge 20.

Typically, a light source (not shown) is connected to the bridge or bridge member 20 to illuminate a fiber optic which extends from the sheath into the urinary organ.

A lens 30 is either removably mounted or integrally formed with the bridge via a hollow instrument channel 32 to enable the urologist to view the organ into which the working end or insertion portion 12 of the cystoscope is inserted.

Distilled water or similar fluid is introduced via an inlet which is controlled with a stopcock 34 for cleansing a urinary bladder.

Referring now to FIG. 2 there is depicted therein a cystoscope having a removable bridge in accordance with the present invention, generally, denoted at 110.

The cystoscope hereof includes a sheath 112 having a proximal end 114 and a distal end 116. The sheath is a hollow tube 115 in which may be provided, at its proximal end, a terminus of a fiber optic 117 or similar means for illuminating an organ within the urinary tract which is under investigation, as detailed below.

It is to be understood, as with the typical prior art cystoscopes, that various instruments may be inserted through the sheath into the desired portion of the urinary tract to perform the requisite procedure, as explained hereinafter.

As shown, the bridge 120 comprises a hollow cylindrical element or tube 150 having a first or connecting end 140 which end detachably or removably connects to the sheath and an opposite or free end 145. The bridge 120 is removably mounted to the proximal end 114 of the sheath 112 by any suitable means, such as a threaded connector 121. Similarly, the bridge 120, other than being threadably attached, may be frictionally fitted to the sheath. Preferably, the end 140 is threaded in complementary manner to a threading provided on the proximal end 114 of the sheath.

The bridge 120 is in registry with and in fluid communication with the sheath.

The bridge tube 150 is fitted with an irrigation tube or tubular element 130 to which an irrigation fluid source tube or the like (not shown) may be fitted for the introduction of cleansing or irrigation fluids from a source through the sheath and to the organ. The source tube may be fitted with a stopcock or similar device for controlling the amount of irrigation fluid introduced into the tube 130.

An observation port 135 is provided at the free end 145 of the bridge. The port 135 removably accommodates a plurality of devices such as an aspirator, diaphragm or the like.

The bridge 120 further includes an offset lens assembly 122 including an eyepiece 122′. The assembly 122 comprises a tubular channel 123, having first and second ends 124, 126, respectively. The channel 123 is integrally formed with the bridge tube 150 and opens into communication with the tube interior at the first end 124 of the channel. The lens or eyepiece 122′ is provided at the opposite or free end 126 of the channel 123.

Suitable minors (not shown) are disposed within the channel 123 to enable the eyepiece to observe the interior of the organ under inspection.

The fiber optic 117, which is either a flexible or rigid bier and, preferably, a flexible fiber is connected to a light source (not shown). The optic, preferably, is fixed in the channel at a first end thereof and, extends from the lens assembly into the bridge tube, and along the length of the sheath and into the organ to be examined in the well-known manner.

The lens assembly 122, as noted, permits the observation of a procedure while it is being conducted. The fiber optic may be disposed in the channel by any suitable means including being fitted directly to the eyepiece which, in turn, is connected directly or indirectly to the light source for rendering the fiber optic operative.

As noted, optimally, the bridge may include an irrigation fluid inlet 130 through which distilled water or other similar fluid is injected into the urinary tract via the sheath 112.

A port (not shown) is provided on the bridge which is used to evacuate any fluid.

As noted, the instrument or observation port 135 removably mounts any desired device including, inter alia, an aspirator for removing particles, or the like.

A secondary light source (not shown), if required, may be provided at the distal end 116 of the sheath to illuminate internal tissue for image capture by a camera device which may be disposed therewithin enabling a surgeon-operator to visually operate a suctioning instrument within the field-of-view.

Similarly, the bridge or the cystoscope utilizing the bridge may be connected to a computer, a monitor, a network or similar electronic means for displaying the procedure as well as storing data generated by the procedure as well.

Having, thus, described the invention what is claimed is:

1. A cystoscope, comprising:
(a) a hollow sheath having a proximal end and a distal end;
(b) a bridge comprising a hollow tube and having a first end and a free end, the bridge being removably mounted to the proximal end of the sheath at its first end and in communication with the sheath, the bridge including:
(1) a fluid irrigation tube follows the bridge and in fluid communication with the sheath;
(2) an offset lens assembly being angularly inclined with respect to the longitudinal axis of the bridge, the assembly including a channel having a first end in communication with the bridge and an opposite free end, and
wherein the offset assembly enables the contemporaneous observation and conducting of a urological procedure.

2. The cystoscope of claim 1, further comprising:
an eyepiece disposed at the free end of the lens assembly; and
a fiber optic fitted to the eyepiece and extending from the lens assembly into the bridge and along the length of the sheath.

3. A removable bridge for use with a cystoscope sheath for conducting urological procedures, comprising:
(a) a hollow tube having a first end and a free end, the tube being removably mounted at its first end to the proximal end of a cystoscopic sheath the tube being in registry with the sheath, the bridge including:
(1) a fluid irrigation tube integral with the bridge tube and in fluid communication with the sheath;
(2) an offset lens assembly being angularly inclined with respect to the longitudinal axis of the bridge tube, the assembly including a channel having a first end in registry and communication with the bridge tube and an opposite free end;
(3) an observation port provided at the free end of the bridge tube;
(b) means for removably attaching the bridge to the sheath,
wherein the offset assembly and observation port cooperate to enable and the contemporaneous observation and conducting of a urological procedure.

4. The cystoscope of claim 3, further comprising:
an eyepiece disposed at the free end of the lens assembly; and
a fiber optic fitted to the eyepiece and extending from the lens assembly into the bridge and along the length of the sheath.

5. A removable bridge for use with a cystoscope sheath for conducting urological procedures, comprising:
(a) a bridge comprising a hollow tube and having a first end and a free end, the tube being removably mounted at its first end to the proximal end of the sheath and in communication with the sheath, the bridge including:
(1) a fluid irrigation tube integral with the bridge tube and in fluid communication with the sheath;
(2) an offset lens assembly being angularly inclined with respect to the longitudinal axis of the bridge tube and, the assembly including a channel integral with the bridge tube and having a first end in communication with the bridge tube and an opposite free end and an eyepiece disposed at the free end of the channel;
(3) an observation port provided at the free end of the bridge tube;
(b) a fiber optic fitted to the eyepiece and extending from the lens assembly into the bridge tube and along the length of the sheath;
(c) means for removably attaching the bridge to the sheath, and
wherein the offset assembly cooperates with the observation port to enable the contemporaneous observation and conducting of a urological procedure.
